(19) United States
(12) Patent Application Publication
(10) Pub. No.: US 2024/0321465 A1
(43) Pub. Date: Sep. 26, 2024

(54) Machine Learning Platform for Predictive Malady Treatment

(71) Applicant: MCKINSEY & COMPANY, INC., New York, NY (US)

(72) Inventors: Maren Eckhoff, London, NY (US); Christoforos Anagnostopoulos, Athens (GR)

(21) Appl. No.: 18/612,790

(22) Filed: Mar. 21, 2024

Related U.S. Application Data
(60) Provisional application No. 63/454,040, filed on Mar. 22, 2023.

(51) Int. Cl. G16H 50/70; G16H 10/60

(52) U.S. Cl. CPC G16H 50/70; G16H 10/60

(57) ABSTRACT

Methods and systems for classifying or predicting maladies that may be treatable by pharmaceuticals, treatments, or procedures. Machine learning models may be trained using one or more sets of patient data to detect connections and patterns between maladies, pharmaceuticals, treatments, tests, etc. to predict potential maladies that might be treatable by a particular pharmaceutical, treatment, and/or procedure.

### CROSS REFERENCE TO RELATED APPLICATIONS

The present application claims the benefit of U.S. Provisional Patent Application No. 63/454,040, titled “Machine Learning Platform for Predictive Malady Treatment”, filed on Mar. 22, 2023, which is hereby expressly incorporated by reference herein in its entirety.

### TECHNICAL FIELD

The present disclosure generally relates to machine learning algorithms, techniques, platforms, methods, and systems for classifying or predicting maladies that may be treatable by pharmaceuticals, treatments, and/or procedures.

### BACKGROUND

Traditionally, predicting whether a pharmaceutical has any effect on a particular malady prior to clinical testing could only be performed using pre-clinical data such as animal models, or computational simulations of the action of the pharmaceutical. However, such predictions are often invalidated upon subsequent clinical testing. In particular, there was no way to leverage the aggregation of real-world data of pharmaceuticals, treatments, procedures, and/or lab tests across a plethora of patients to accurately predict which, if any, malady might potentially be treatable by a pharmaceutical, treatments, and/or procedure.

Accordingly, herein, in order to address the aforementioned issues, systems and methods for classifying or predicting maladies that may be treatable by pharmaceuticals, treatments, and/or procedures are disclosed.

### SUMMARY

In some embodiments, an artificial intelligence based method for classifying or predicting maladies that may be treatable by pharmaceuticals, treatments, and/or procedures may be provided. The method may be implemented via one or more local or remote processors, servers, memory units, mobile devices, wearables, and/or other electronic or electrical components. In various aspects, the techniques described herein relate to an artificial intelligence based method for classifying or predicting maladies that may be treatable by pharmaceuticals, treatments, and/or procedures, the artificial intelligence based method including: receiving, by one or more processors, one or more sets of patient data; determining, by the one or more processors, one or more clinical events in the one or more sets of patient data; applying, by the one or more processors, a first developed machine learning model on the one or more clinical events to generate a set of clinical event representations; applying, by the one or more processors, a second developed machine learning model on the set of clinical event representations to generate a set of similarities; filtering, by the one or more processors based upon one or more predetermined clinical events, the set of similarities to generate a set of similarity scores; and presenting, by the one or more processors, at least a portion of the set of similarities to a client device. The method may include additional, less, or alternate actions, including those discussed elsewhere herein.

In other embodiments, a computer system for classifying or predicting maladies that may be treatable by pharmaceuticals, treatments, and/or procedures may be provided. The computer system may include, or be configured to work with, one or more local or remote processors, servers, memory units, mobile devices, wearables, and/or other electronic or electrical components. In various aspects, the techniques described herein relate to a computer system for classifying or predicting maladies that may be treatable by pharmaceuticals, treatments, and/or procedures, the computer system including: one or more processors; and one or more non-transitory program memories coupled to the one or more processors, the one or more memories storing executable instructions that, when executed by the one or more processors, cause the one or more processors to: receive one or more sets of patient data; determine one or more clinical events in the one or more sets of patient data; apply a first developed machine learning model on the one or more clinical events to generate a set of clinical event representations; apply a second developed machine learning model on the set of clinical event representations to generate a set of similarities; filter, based upon one or more predetermined clinical events, the set of similarities to generate a set of similarity scores; and present at least a portion of the set of similarities to a client device. The computer system may be configured to include additional, less, or alternate functionality, including that discussed elsewhere herein.

In yet other embodiments, a non-transitory computer-readable medium for classifying or predicting maladies that may be treatable by pharmaceuticals, treatments, and/or procedures may be provided. Executable instructions may be stored on the non-transitory computer-readable medium for classifying or predicting maladies that may be treatable by pharmaceuticals, treatments, and/or procedures, the instructions, when executed by one or more processors, may cause the one or more processors to: receive one or more sets of patient data; determine one or more clinical events in the one or more sets of patient data; apply a first developed machine learning model on the one or more clinical events to generate a set of clinical event representations; apply a second developed machine learning model on the set of clinical event representations to generate a set of similarities; filter, based upon one or more predetermined clinical events, the set of similarities to generate a set of similarity scores; and present at least a portion of the set of similarities to a client device. The instructions may direct additional, less, or alternate functionality, including that discussed elsewhere herein.

The present disclosure may include improvements in computer functionality or in improvements to other technologies at least because the disclosure herein discloses systems and methods for classifying or predicting maladies that may be treatable by pharmaceuticals, treatments, and/or procedures. The systems and methods herein may train machine learning models using input data (e.g., one or more sets of patient data, etc.) to predict whether a malady might be affected by a particular pharmaceutical, treatment, and/or procedure based upon the relationship between various clinical events in the patient data. For example, when deployed on the underlying system, the machine learning models allow the systems and methods of the present disclosure to execute with fewer iterations, and use fewer computing resources, than prior art related systems and methods, at least because such prior art systems would require manual data entry, data storage, and/or implementation, all of which result in greater memory usage and processor utilization. Additional improvements may also include determined corollary and/or causal outputs. For example, the system, utilizing the machine learning models, may be able to explain why a certain indication is ranked high with regard to a different referential.

Similarly, the present disclosure describes improvements in the functioning of the computer itself or “any other technology or technical field” because the data generated (e.g., the predicted maladies that might be treatable by a pharmaceutical, treatment, and/or procedure) described herein allows the underlying computer system to utilize less processing and memory resources compared to prior art systems and methods. This is at least because the use of the machine learning models results in fewer compute cycles, or otherwise iterations, that has less of an impact on the underlying computing device compared to previous prior art systems and methods.

In addition, the present disclosure relates to improvement to other technologies or technical fields at least because the systems and methods of the present disclosure provide a robust, efficient, and comparable model that can be used to improve the efficiency and performance of several downstream pharmaceutical discovery, development, and/or manufacturing tasks. This may be performed, for example, by a machine learning model that is determined or otherwise generated based upon determined interconnected relationships between the various clinical events in the patient data. The machine learning model may be deployed on an underlying computing device or system, thereby, improving its accuracy and prediction in performing pharmaceutical discovery, development, and/or manufacturing tasks as described herein.

Still further, the present disclosure includes specific features other than what is well-understood, routine, conventional activity in the field, and/or otherwise adds unconventional steps that confine the disclosure to a particular useful application (e.g., systems and methods for predicting a malady that might be treatable by a pharmaceuticals, treatment, and/or procedure based upon the machine learning model) which can be used, for example, for the effective and efficient output of determining whether a pharmaceutical, treatment, and/or procedure will have a likely effect on a malady, which may be used or applied for pharmaceutical discovery, development, and/or manufacturing applications. Example applications may include: prioritizing which maladies, pharmaceuticals, treatments, and/or procedures to further research in a clinical development strategy plan; determining which pharmaceuticals, treatments, and/or procedures should receive further clinical testing in order to determine whether known pharmaceuticals, treatments, and/or procedures might be a potential treatment of a malady; and/or the like.

Advantages will become more apparent to those of ordinary skill in the art from the following description of the preferred embodiments, which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The FIGs. described below depict various embodiments of the systems and methods disclosed herein. It should be understood that the FIGs. depict illustrative embodiments of the disclosed systems and methods, and that the FIGs. are intended to be exemplary in nature. Further, wherever possible, the following description refers to the reference numerals included in the following FIGs., in which features depicted in multiple FIGs. are designated with consistent reference numerals.

There are shown in the drawings arrangements which are presently discussed, it being understood, however, that the present embodiments are not limited to the precise arrangements and instrumentalities shown, wherein:

FIG. 1 depicts exemplary components, apparatuses, and devices used by devices and systems for implementing a malady-treatment prediction system;

FIG. 2 depicts an exemplary computing environment including components, apparatuses, and devices for implementing the malady-treatment prediction system;

FIG. 3 depicts an exemplary workflow of the malady-treatment prediction system;

FIG. 4A depicts exemplary sets of patient data of the malady-treatment prediction system;

FIG. 4B depicts an exemplary set of clinical event representations of the malady-treatment prediction system;

FIG. 4C depicts an exemplary set of similarities of the malady-treatment prediction system;

FIG. 4D depicts an exemplary set of similarity scores of the malady-treatment prediction system;

FIG. 4E depicts exemplary output data of the malady-treatment prediction system;

FIG. 5 depicts exemplary machine learning modules;

FIG. 6 depicts an exemplary flowchart representative of example methods, hardware logic, and instructions for implementing the malady-treatment prediction system;

FIG. 7 depicts an exemplary flowchart representative of example methods, hardware logic, and instructions for implementing the machine learning training modules; and

FIG. 8 depicts an exemplary computer-implemented method for classifying or predicting maladies that may be treatable by pharmaceuticals, treatments, and/or procedures

The figures depict the present embodiments for purposes of illustration only. One skilled in the art will readily recognize from the following discussion that alternate embodiments of the structures and methods illustrated herein may be employed without departing from the principles of the invention described herein.

### DETAILED DESCRIPTION

Embodiments of the present description relate to computing systems and methods for classifying or predicting maladies that may be treatable by pharmaceuticals, treatments, and/or procedures. Herein, maladies, pharmaceuticals, treatments, and procedures are defined by their broadest possible definitions. For example, maladies may refer to one or more infectious diseases, one or more deficiency diseases, one or more hereditary diseases, one or more physiological diseases and/or conditions, physical conditions and/or disorders, mental and/or neurological disorders, cancers, disabilities, etc. As another example, maladies may refer to specific strands of viruses, bacteria, and/or multicellular organisms (such as parasites) infecting individuals and/or any other illness, disease, and/or condition capable of a diagnosis and/or a prognosis (even if such a diagnosis and/or prognosis does not yet exist in the various health fields) as well as specific severities and/or progression stages of such illnesses, diseases, and/or conditions.

By leveraging a large collection of real-world data (e.g., diagnosis of maladies, prescription of pharmaceuticals, performance of procedures, conduction of lab tests, etc. on a multitude of patients), otherwise unseen connections between various clinical events may discovered. The machine learning models may be trained to discover these connections which in turn may allow one or more methods and/or systems, utilizing machine learning models, to predict which maladies may be treatable by a pharmaceutical, treatment, and/or procedure.

To make the resulting models be as accurate as possible, it is recommended that large data sets of patients be collected with as much detail as possible. As an example, a data set of a million patients each featuring their complete medical history with dates and timelines may be gathered and/or analyzed by the machine learning models. Aspects of the various clinical events may be gathered and be used in the calculation (e.g., a chronic disease that is continuously diagnosed in a patient may be weighted greater than a disease that was diagnosed only once and easily treated). However, the data sets need not be large in order to achieve accurate results. For example, a data set of one thousand patients, each patient featuring a complete medical history with additional granularity on specific biomarkers, may be sufficient in training, validating, and/or utilizing the models described herein.

### Exemplary Machine Learning Techniques

The present embodiments may involve, inter alia, the use of cognitive computing, predictive modeling, machine learning, causal inference and/or other modeling techniques and/or algorithms. In particular, one or more sets of patient data and/or the like may be input into one or more machine learning programs described herein that are trained and/or tested to classifying or predicting maladies that may be treatable by pharmaceuticals, treatments, and/or procedures.

In certain embodiments, the systems, methods, and/or techniques discussed herein may use heuristic engines, algorithms, machine learning, cognitive learning, deep learning, combined learning, predictive modeling, and/or pattern recognition techniques. For instance, a processor and/or a processing element may be trained using supervised, unsupervised, and/or semi-supervised machine learning, and the machine learning program may employ a neural network, which may be a convolutional neural network, a deep learning neural network, and/or a combined learning module or program that learns in two or more fields or areas of interest. Machine learning may involve identifying and/or recognizing patterns in existing data in order to facilitate making predictions, estimates, and/or recommendations for subsequent data. Models may be created based upon example inputs in order to make valid and reliable predictions for novel inputs.

Additionally or alternatively, the machine learning programs may be trained and/or tested by inputting sample data sets or certain data into the programs, such as one or more sets of patient data and/or known resulting data (e.g., an excluded portion of the one or more sets of patient data). The machine learning programs may utilize deep learning algorithms that may be primarily focused on pattern recognition and may be trained after processing multiple examples. The machine learning programs may also include natural language processing, semantic analysis, automatic reasoning, and/or machine learning.

In supervised machine learning, a processing element may be provided with example inputs and their associated outputs. The processing element may seek to discover a general rule that maps inputs to outputs, so that, when subsequent novel inputs are provided, the processing element may, based upon the discovered rule, accurately predict the correct output. In unsupervised machine learning, the processing element may be required to find its own structure in unlabeled example inputs. In semi-supervised machine learning, the processing elements may use thousands of individual supervised machine learning iterations to generate a structure across the multiple inputs and outputs.

### Exemplary Components, Apparatuses, and Devices

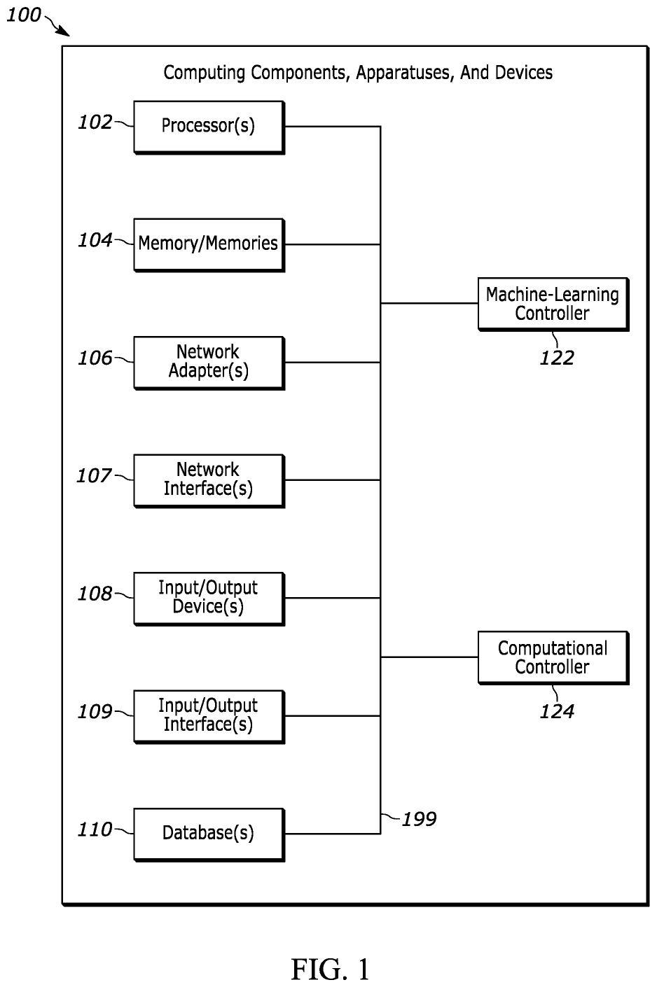

FIG. 1 depicts a block diagram of exemplary components, apparatuses, and devices 100 to classifying or predicting maladies that may be treatable by pharmaceuticals, treatments, and/or procedures.

The exemplary components, apparatuses, and devices 100 may include one or more processors 102 (e.g., a programmable processor, a programmable controller, a CPU, a GPU, a DSP, an ASIC, a PLD, an FPGA, an FPLD, a spark cluster, etc.), one or more memories (e.g., random access memory (RAM), read only memory (ROM), cache, etc.) 104, one or more network adapters 106, one or more network interfaces 107, one or more I/O devices 108, one or more I/O interfaces 109, one or more databases 110, one or more machine-learning controllers 122, and/or one or more computational controllers 124 all of which may be interconnected via an address/data bus 199. The one or more memories 104 may store software and/or computer-executable instructions, which may be executed by the one or more processors 102.

The one or more processors 102 may be, or may include, a central processing unit (CPU), a graphical processing unit (GPU), a digital signal processor (DSP), an application specific integrated circuit (ASIC), a programmable logic device (PLD), a field-programmable gate array (FPGA), a field-programmable logic device (FPLD), etc.

The one or more memories 104 may be, or may include, any local short term memory (e.g., random access memory (RAM), read only memory (ROM), cache, etc.) and/or any long term memory (e.g., hard disk drives (HDD), solid state drives (SSD), cloud storage, etc.).

The one or more network adapters 106 and/or the one or more network interfaces 107 may be, or may include, a wired network adapter, connector, interface, etc. (e.g., an Ethernet network connector, an asynchronous transfer mode (ATM) network connector, a digital subscriber line (DSL) modem, a cable modem) and/or a wireless network adapter, connector, interface, etc. (e.g., a Wi-Fi connector, a Bluetooth® connector, an infrared connector, a cellular connector, etc.).

The one or more I/O devices 108 may be, or may include, any number of different types of peripheral devices for either inputting data or outputting results. The peripheral devices may be any desired type of device such as a keyboard, a display (a liquid crystal display (LCD), a cathode ray tube (CRT) display, touch, etc.), a navigation device (a mouse, a trackball, a capacitive touch pad, a joystick, etc.), a speaker, a microphone, a button, a communication interface, an antenna, etc. The one or more I/O interfaces 109 may include any number of different types of input and/or output units and/or combined I/O circuits and/or components that enable the one or more processors 102 to communicate with the peripheral devices.

The one or more databases 110 may be a server or some other form of data storage device (e.g., one or more memories 104, CDs, CD-ROMs, DVDs, Blu-ray disks, layers and/or parquet files of a cloud storage network, etc.). In some examples, the one or more databases 110 store one or more sets of training/testing data.

The one or more machine-learning controllers 122 and/or the one or more computational controllers 124 may be, or may include, computer-readable, executable instructions that may be stored in the one or more memories 104 and/or performed by the one or more processors 102. Further, the computer-readable, executable instructions of the one or more machine-learning controllers 122 and/or the one or more computational controllers 124 may be stored on and/or performed by specifically designated hardware (e.g., micro controllers, microchips, etc.) which may have functionalities similar to the one or more memories 104 and/or the one or more processors 102.

### Exemplary Machine Learning Environments

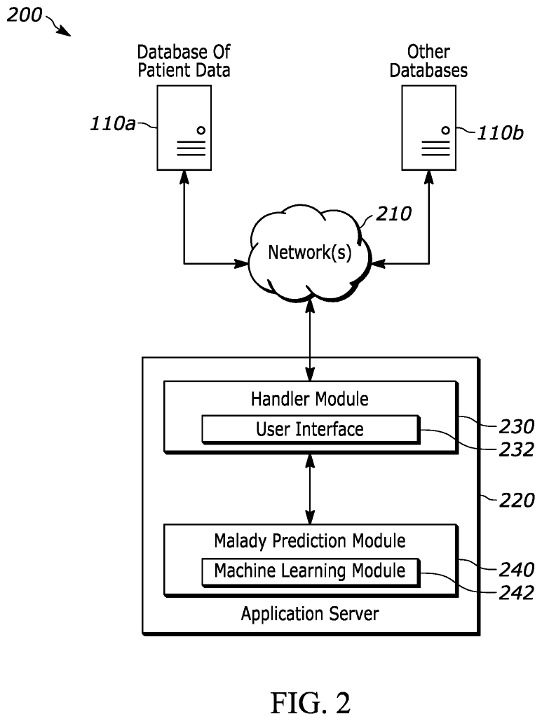

FIG. 2 depicts a diagram of an exemplary computing environment 200. The computing environment 200 may include one or more databases of patient data 110a, one or more other databases 110b, one or more networks 210, an application server 220, a handler module 230, a user interface (UI) 232, a malady prediction module 240, and/or a machine learning modules 242.

The one or more databases of patient data 110a and/or the one or more other databases 110b may be, or may include, one or more databases, servers, data repositories, etc. (e.g., the one or more databases 110). The one or more networks 210 may be, or may include, the internet, a local area network (LAN), a metropolitan area network (MAN), a wide area network (WAN), a wired network, a Wi-Fi network, a cellular network, a wireless network, a private network, a virtual private network, etc.

The application server 220 may include the handler module 230, and/or the malady prediction module 240. The handler module 230 may include UI 232. The malady prediction module 240 may include machine learning modules 242. The application server 220, the handler module 230, the UI 232, the malady prediction module 240, and/or the machine learning modules 242, may be, or may include, a portion of a memory unit (e.g., the one or more memories 104 of FIG. 1) configured to store software and/or computer-executable instructions that, when executed by a processing unit (e.g., the one or more processors 102 of FIG. 1), may cause the one or more of the aforementioned components to classifying or predicting maladies that may be treatable by pharmaceuticals, treatments, and/or procedures.

In operation, the application server 220 may connect to the one or more databases, servers, and/or other data repositories (e.g., one or more databases of patient data 110a and/or the one or more other databases 110b, etc.) via one or more networks 210. In some embodiments, the connection may include a client device establishing a client-host connection to the application server 220. In these embodiments, client device may establish the client-host connection via an application run on the client device. In some embodiments, the connection may be through either a third party connection (e.g., an email server) or a direct peer-to-peer (P2P) connection/transmission.

The handler module 230 may receive one or more sets of input data over the one or more networks 210. The handler module 230 may forward the one or more sets of input data to the malady prediction module 240. The malady prediction module 240 may pass the one or more sets of input data through the machine learning modules 242, which may generate one or more classified or predicted maladies that may be treatable by a pharmaceutical, treatment, and/or procedure. The one or more one or more classified or predicted maladies that may be treatable by a pharmaceutical, treatment, and/or procedure may be returned to the handler module 230 which may in turn present the classified or predicted maladies to a user of the client device.

In some embodiments, the handler module 230 may implement an interactive UI 232 (e.g., a web-based interface, mobile application, etc.) that may be used by the user of the client device to receive one or more one or more classified or predicted maladies that may be treatable by a pharmaceutical, treatment, and/or procedure. For example, the interactive UI 232 may be utilized to review the validation of the machine learning model to accurately generate confidence values in the resulting similarity scores and ranks. As another example, the interactive UI 232 may allow a user to view portions of the generated ranked outputs (e.g., view the top 30 ranked clinical events, view the top 100 ranked clinical events, view the clinical events with a rank between 30-100, etc.). As yet another example, the interactive UI 232 may be used to search through the resulting output to find the rank of a particular clinical event.

The machine learning modules 242 may generate a machine learning model based upon training data. The training data may include one or more sets of patient data. The one or more sets of patient data may include a set of characteristics of each patient including other data (e.g., demographics of the patient, medical history of the patient, diagnosis of a malady, length of time a malady was contracted, prescription of a pharmaceutical, length of time a pharmaceutical was prescribed, dates of lab tests and their results, etc.). In some embodiments, the training data may be data separate from the input data (e.g., curated training data, simulated training data, and/or input data from previous training and/or applications of the machine learning model). Additionally or alternatively in some embodiments, the training data may be the input data to be analyzed by the machine learning model.

The machine learning modules 242 may utilize unsupervised learning and/or semi-supervised learning to determine one or more unlabeled categorizations, classifications, dimensions, and/or parameters of the input data. In some aspects, other types of machine learning techniques may be utilized, such as semi-supervised learning methods and attention methods, dimensionality reduction, anomaly detection, low-density separation, Laplacian regularization, neural networks, deep learning, and/or any other suitable machine learning technique.

For example, when the machine learning technique is dimensionality reduction, the machine learning modules 242 may determine one or more dimensions and/or parameters of the input data for each clinical event identified within the one or more sets of patient data. This collection of identified clinical events is referred to herein as the set of clinical events, and the set of clinical events with one or more dimensions and/or parameters for each clinical event is referred to herein as a set of clinical event representations.

As another example, when the machine learning model is developed via semi-supervised learning, the machine learning modules 242 may also characterize the input data into a set of clinical event representations. From this characterization, the machine learning modules 242 may then determine a plurality of relationships and contexts between the various clinical events. The machine learning modules 242 may hide one or more clinical events and/or contexts (e.g., an amount of time between clinical events) and then attempt to predict the hidden clinical event and/or context based upon the previously determined relationships in the input data. In doing so, the machine learning modules 242 may determine and assign various weights for each determined relationship. Once developed, the machine learning model may be able to predict clinical events based upon given contexts, predict contexts based upon given clinical events, and/or determine the likelihood of a clinical event and context both occurring in a patient's medical history. Thus, the clinical event representations may be derived from the generated weights of the machine learning modules 242.

Once the categorizations, classifications, dimensions, and/or parameters of the input data have been determined, the machine learning model may perform a distance and/or similarity algorithm on the one or more clinical events based upon the determined categorizations, classifications, dimensions, and/or parameters. For example, the machine learning modules 242 may perform pair-wise similarity between each of the clinical event representations of each patient in input data to determine a set of similarities between each of the clinical events. Any appropriate distance and/or similarity algorithm may be utilized to generate the set of similarities, such as cosine similarity, Manhattan distance, Euclidian distance, Jaccard similarity, Dice similarity, and/or the like.

The machine learning modules 242 may validate the resulting machine learning model generated. In some embodiments, this may be performed by obtaining stability and/or confidence estimates around the scores and/or ranks by iteratively running the machine learning model multiple times on different slices of the data and changing random seeds (e.g., varying the probabilistic aspects of the machine learning model). For example, confidence estimates for each malady may be obtained by analyzing the distribution of that malady's score and/or rank across the different iterative runs. In one embodiment, the malady rankings may be penalized if the variation across different iterative runs is greater than a threshold value.

Once the machine learning model has been sufficiently trained and/or validated, malady prediction module 240 may use the machine learning model to prioritize the clinical events from the resulting output. The malady prediction module 240 may then use the resulting outputs to predict one or more maladies that have the highest likelihood of being affected by the particular pharmaceutical, treatment, and/or procedure. The malady prediction module 240 may return the results to the handler module 230. The handler module 230 may pass the results to the user of the client device.

It should be appreciated that while specific elements, processes, devices, and/or components are described as part of the application server 220, other elements, processes, devices and/or components are contemplated.

### Exemplary Input Data

Example input data may include input data such as one or more sets of patient data and/or other data.

The one or more sets of patient data may include (a) a set of characteristics of each patient (e.g., demographics of the patient, medical history of the patient, etc.), (b) a set of previously contracted maladies, the number of occurrences of each contracted malady, the date of diagnosis of each malady in each patient, the specialty of the healthcare professional diagnosing the maladies, etc., (c) a set of pharmaceuticals prescribed and/or given to each patient, the date the pharmaceutical was prescribed and/or taken, the dosage of the pharmaceutical, the length of time of treatment using the pharmaceutical, etc. (d) a set of procedures performed on each patient and/or date the procedure was performed, (e) a set of lab tests and their corresponding results performed on each patient, the lab tests' date of performance, and/or the lab results' date of reporting, (f) a set of clinical observations (e.g., visits to specialists such as dermatologists, neurologists, etc.) and/or the date those clinical observations were made, (g) a set of emergency medical treatments (e.g., from EMTs, hospital emergency rooms, etc.) and/or the date those emergency medical treatments were made, (h) a set of monitored data (e.g., from an electrocardiogram, a wearable, and/or the like), and/or like data. The one or more sets patient data may include and/or exclude any of the foregoing exemplary data and/or subsets of the foregoing exemplary data (e.g., only diagnosis of neurological disorders are included, and/or all diagnosed cancers are excluded, and/or Asthma and breast cancer is included, and/or only variants of coronavirus are included, and/or abnormal FEV1 test results are included, etc.).

Any of the foregoing example input data may be determined by the components, apparatuses, and devices 100 and/or the computing environment 200 as determined data and/or received by the components, apparatuses, and devices 100 and/or the computing environment 200 from one or more databases, servers, and/or other data repositories (e.g., one or more databases of patient data 110a and/or the one or more other databases 110b, etc.) over one or more networks as received data.

The components, apparatuses, and devices 100 and/or the computing environment 200 may determine any of the aforementioned data and/or any other data based upon preexisting data. For instance, the components, apparatuses, and devices 100 and/or the computing environment 200 may determine the severity of a diagnosed malady by correlating the one or more diagnoses of the malady in a patient to one or more results of lab tests performed on the patient.

The components, apparatuses, and devices 100 and/or the computing environment 200 may receive any of the aforementioned data and/or any other data from one or more databases and/or other data repositories stored across one or more networks 210.

Any of the foregoing input data may include one or more fields, labels, entries, parameters, and/or values in addition to, interchanged with, and/or instead of those listed.

### Exemplary Application Using Dummy Data

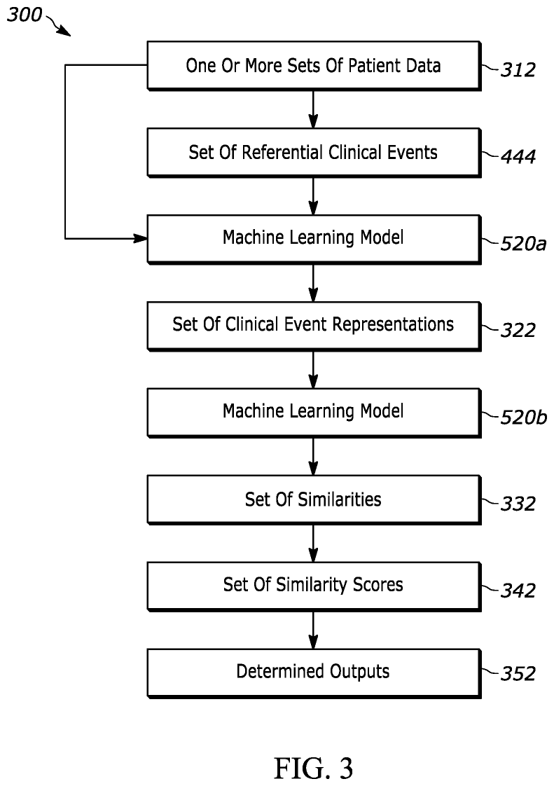

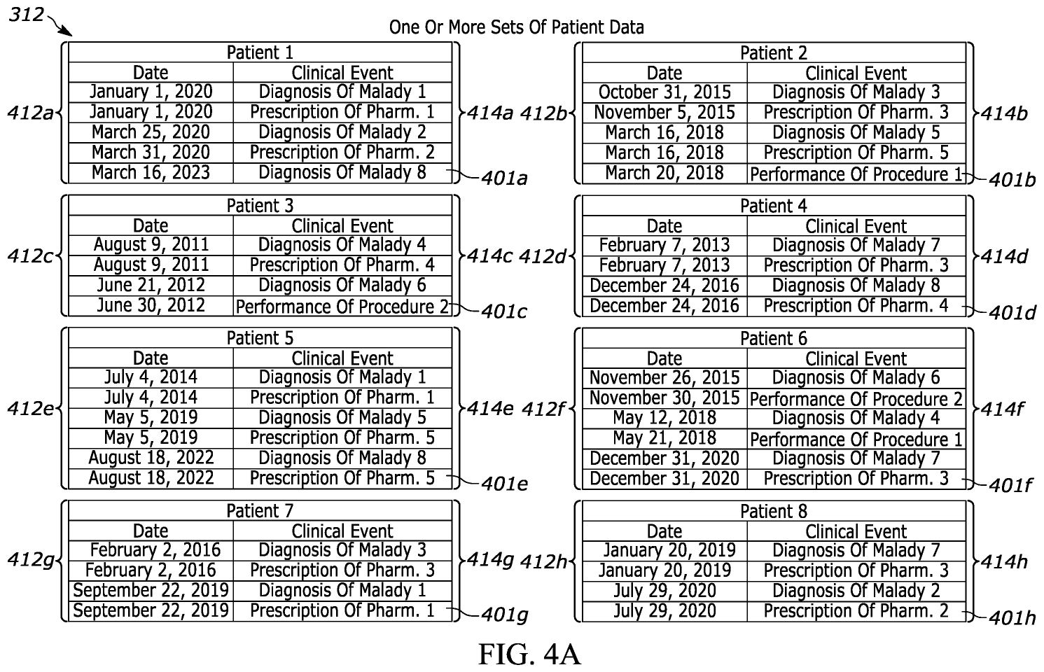

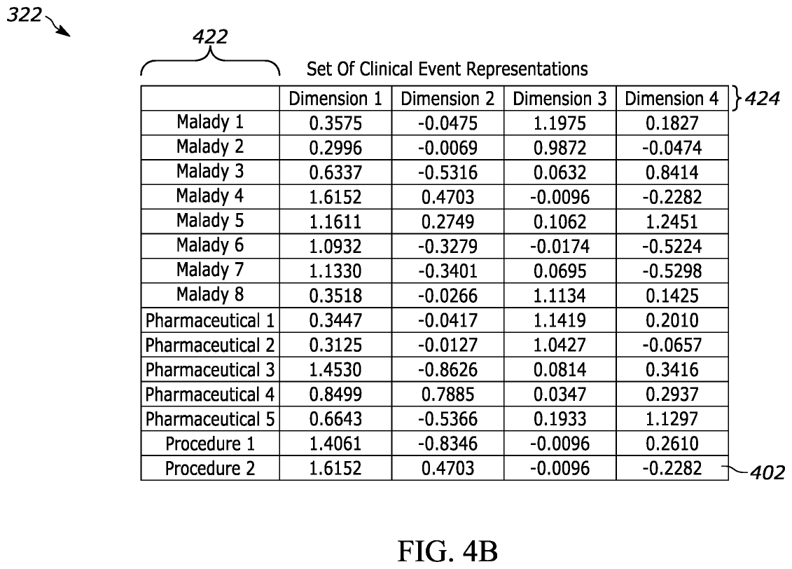

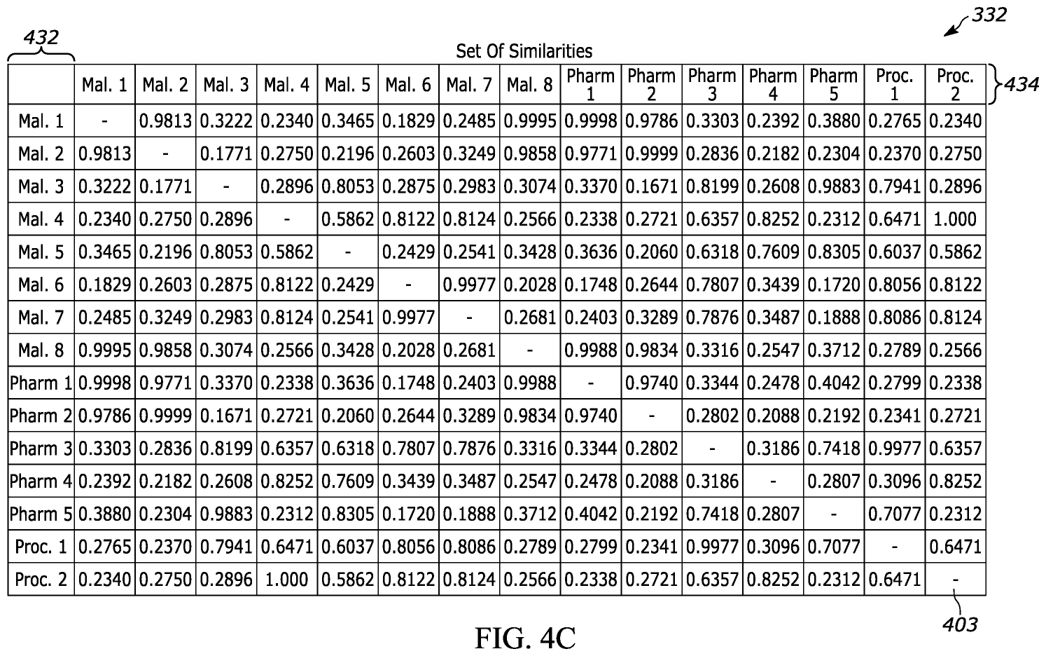

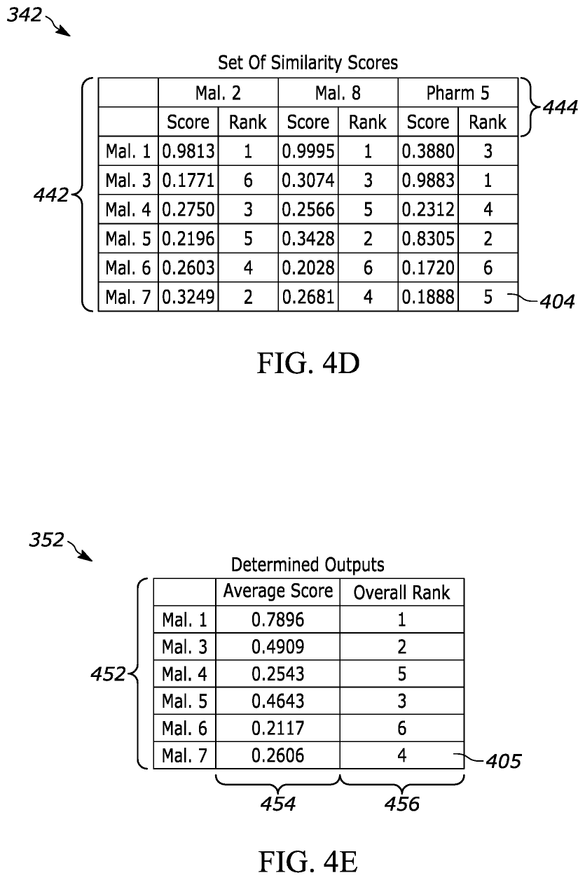

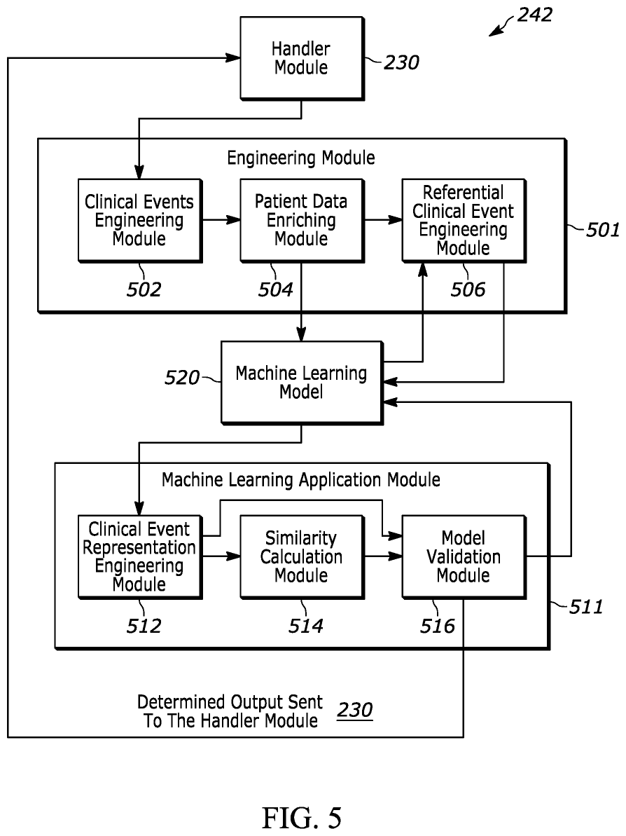

Prior to application, the malady-treatment prediction system may first generate an input vector of the machine learning model 520 using the input data described above. The input vector may include at least one two-dimensional (2D) set (e.g., lists, tables matrices, etc.) of data, as illustrated in FIGS. 3-4E. In various aspects, the data may comprise pre-processing, formatting, and/or normalizing data into different formats, representations, or values in order to improve the machine learning model 520, for example, by making its output more accurate and/or by reducing the data input size and/or amount needed for executing the machine learning model 520 once trained.

In some embodiments, the input vector may be derived by the processing one or more sets of patient data 312. The one or more sets of patient data 312 may be one or more 2D subsets of data corresponding to each patient in the one or more subsets of patient data 312 where a first column of each subset indicates one or more clinical events (e.g., a diagnosis of a malady, a prescription of a pharmaceutical, a performance of a procedure, a performance of a lab test, etc.) 414a-h and a second column of each subset indicates a date of the clinical event 412a-h as illustrated in FIG. 4A. In many embodiments, the individual entries in each subset may connect a date to a clinical event (e.g., Patient 1 was diagnosed with Malady 1 on Jan. 1, 2020) 401. It should be noted that while FIG. 4A features eight patient subsets, the one or more sets of patient data may include any number of subsets and each subset may include any number of clinical events with corresponding dates and that the clinical events may feature any number of aspects of information (e.g., dosages of pharmaceuticals, etc.). The one or more sets of patient data 312 may be engineered based upon one or more sources of raw, real-world data, and the raw, real-world data may be granular in nature.

In some embodiments, the one or more sets of patient data 312 may be collected, counted, and/or normalized to generate a combined set of patient data (not shown). The combined set of patient data may be a 2D set of data where a first column indicates a patient identifier (e.g., a patient ID code), a second column indicates one or more clinical events (e.g., a diagnosis of a malady, a prescription of a pharmaceutical, a performance of a procedure, a performance of a lab test, etc.), and a third column indicates a date of the clinical event. The individual entries of the combined set of patient data may connect a date to a clinical event (e.g., Patient 1 was diagnosed with Malady 1 on Jan. 1, 2020).

Additionally or alternatively, in some embodiments, the combined set of patient data may be a 2D set of data where both rows and columns indicate one or more clinical events. In these embodiments, the individual entries of the combined set of patient data may indicate a total number of occurrences of a clinical event (e.g., a diagnosis of Malady 1 and Malady 2 co-occurred 34 times within a 6 month window).

It should be noted that the one or more sets of patient data 312 illustrated in FIG. 4A is just an example embodiment and that more, less, and/or alternative data points may be used in the description herein.

In some embodiments, the one or more sets of patient data 312 and/or the combined set of patient data may be fed into a first machine learning model (e.g., machine learning model 520a) to generate a set of clinical event representations 322. The set of clinical event representations 322 may be a 2D set of data where the rows list the distinct clinical events and the columns indicate the determined dimensions of clinical event representations learned from the one or more sets of patient data 312 and/or the combined set of patient data. The individual entries of the set of clinical event representations 322 indicate determined categorizations, classifications, dimensions, and/or parameters of each clinical event in the one or more sets of patient data 312 and/or the combined set of patient data, as illustrated in FIG. 4B. In some embodiments, the set of clinical event representations 322 may be generated using unsupervised and/or semi-supervised machine learning. For example, the machine learning model 520 may perform classical dimensionality reduction (e.g., principal component analysis (PCA), linear discriminant analysis (LDA), etc.), shifted positive pointwise mutual information (SPPMI), and/or any other appropriate machine learning technique (e.g., semi-supervised learning approached leveraging deep learning and self-attention) on the one or more sets of patient data 312 and/or the combined set of patient data to generate the set of clinical event representations 322. Additionally or alternatively, the data in the one or more sets of patient data 312 and/or the combined set of patient data may be weighted. For example, chronic maladies may be given a lower weight than rare maladies, or a pharmaceutical may be weighted higher if it is typically prescribed close in time to a related diagnosed malady (e.g., what the pharmaceutical was prescribed for, strengthening that correlation). As another example, one or more data in the one or more sets of patient data 312 and/or the combined set of patient data may feature sliding windows to adjust weightings (e.g., a patient may be counted multiple times by having a sliding window). To put another way, the system can apply different weights to the different time points (e.g., use patient once and give all events in the journey equal weight, or use patients multiple times by having a sliding window, or use patient multiple times by having a sliding window and weight events further away from the center of this window less).

It should be appreciated that the set of clinical event representations 322 shown is just one illustrative example and that more, less, and/or alternative event representations are considered.

In some embodiments, the set of clinical event representations 322 may be fed into a second machine learning model (e.g., machine learning model 520b) to generate one or more sets of similarities 332. In some embodiments, the second machine learning model is the same as the first machine learning model. The one or more sets of similarities 332 may be a 2D set of data where the rows of the set of similarities 332 indicate one or more distinct clinical events 432, the columns of the set of similarities 332 also indicate the same one or more distinct clinical events 434, and the individual entries of the set of similarities 332 indicate the pair-wise distance and/or similarity between each of the clinical events with one another 403, as illustrated in FIG. 4C. As an example, cosine similarity is performed on the combined set of patient data 322 to generate the individual entries of the set of similarities 332, where each column vector of the combined set of patient data 322 is compared to each other column vector (whereby the total number of occurrences of each clinical event is compared). But it should be appreciated that any appropriate similarity and/or distance function may be applied to the combined set of patient data 322.

Once the set of similarities 332 have been generated, the set of similarities 332 may be filtered to generate a resulting set of similarity scores 342 based upon one or more referential clinical events. The referential clinical events may be determined before applying the machine learning model 502 based upon one or more sources of real-world data (e.g., established drug approvals, clinical knowledge and expertise, medical literature, gene databases, and/or biomedical knowledge graphs). The referential clinical events may be determined based upon a threshold number of evidentiary findings that may indicate connections with a particular pharmaceutical, treatment, and/or procedure. In this example, the referential clinical events are Malady 5, Malady 8, and Pharmaceutical 3, as illustrated in FIG. 4D. The set of similarity scores 342 may be a 2D set of data where the rows of the set of similarity scores 332 indicate the distinct clinical events from the one or more sets of similarities 442 and the columns of the set of similarity scores 332 indicate the referential clinical events 444. In some embodiments, the set of similarity scores 342 may be filtered to only show the similarity scores between the clinical events related to the diagnosis of maladies and the referential clinical events, as illustrated in FIG. 4D. In some embodiments, the referential clinical events 444 may be bifurcated by a score (e.g., the previously determined similarity values of each clinical event to that particular referential clinical event) and a rank (based upon the various similarity values), as illustrated in FIG. 4D. As an example, Malady 3 has a similarity value of “0.9883” to Pharmaceutical 5, as determined in the set of similarities 332 (see FIG. 4C). Therefore, Malady 3 has a score of “0.9883” for Pharmaceutical 5, as illustrated in FIG. 4D. Additionally, because Malady 3 has the highest similarity value among all other clinical events to Pharmaceutical 5, Malady 3 has a Rank of “1” under Pharmaceutical 5, as illustrated in FIG. 4D.

Once the set of similarity scores 342 have been generated, additional processing may be performed to determine outputs 352 based upon overall scoring and/or overall ranking between the similarity scores and/or ranks determined in the set of similarity scores 342. The determined outputs 352 may be a 3D set of data where the rows of the determined outputs 352 indicate the clinical events from the set of similarity scores 342, a first column of the determined outputs 352 indicates a statistical aggregate score and/or rank (e.g., a mean rank, a median rank, an average score, etc.), and a second column of the determined outputs 352 indicates an overall rank. The individual entries of the determined outputs may connect the individual clinical events to the statistical aggregate score and/or rank and the overall rank 405. For example, because Malady 1 has the greatest average score across each of the referential clinical events in the set of similarity scores 342 (with an average score of “0.7896”), Malady 1 may be given an overall rank of Rank “1” and may be predicted by the malady-treatment prediction system as the best candidate to be treatable by the target pharmaceutical, treatment, and/or procedure, as illustrated in FIG. 4E.

### Alternate Exemplary Application Using Dummy Data

As another example, consider the following 2D sets of input data analyzed by the machine learning model. In this example, Example Table 1 may be a set of patient data. A first column of the Example Table 1 may represent different clinical events in a patient's medical history (in this case Patient 1), and a second column of the Example Table 1 may represent the dates a clinical event occurred (e.g., the first entry of Example Table 1 may be interpreted to mean Patient 1 was diagnosed with X1 on Jan. 1, 2020).

EXAMPLE TABLE 1
Patient 1

| Date | Clinical Event |
|---|---|
| Jan. 1, 2020 | Diagnosis X1 |
| Jan. 5, 2020 | Prescription Y |
| Jan. 5, 2020 | Diagnosis X2 |
| Jan. 21, 2020 | Procedure Z |
| Jan. 27, 2020 | Diagnosis X2 |

Example Table 1 may be a portion of a larger 2D set of patient data with the full set of patient data corresponding to Patient 1's entire medical history. One or more sets of patient data may include multiple subsets of patient data wherein each subset corresponds to an individual patient's medical history. For example, a similar table to that of Example Table 1 may correspond to Patient 2, and so on. The one or more sets of patient data may be fed into a machine learning model. The machine learning model may be trained using unsupervised learning techniques (e.g., classical dimensionality reduction, shifted positive pointwise mutual information, etc.) and/or using semi-supervised learning techniques. An example of a semi-supervised learning technique involves hiding portions of each patient's medical history and fit a large parameter model to predict the “hidden” part. To use Example Table 1 as an example, the malady-treatment prediction system may remove or otherwise exclude the data entry of “Jan. 5, 2020-Diagnosis X2.” The machine learning system may use various connections (such as how Procedure Z is typically performed after a diagnosis of X2 or another patient's data to recognize trends and/or patterns to deduce that the hidden data entry should be Diagnosis of X2).

As an example, a counting of occurrences of the various clinical events across each patient may be performed, and the resulting count may be passed through a machine learning model which in turn may perform dimensionality reduction to generate the following table.

EXAMPLE TABLE 2

| Clinical Event | Dimension 1 | Dimension 2 | Dimension 3 | Dimension 4 | Dimension 5 |
|---|---|---|---|---|---|
| Diagnosis x1 | 0.21 | 0.47 | 1.8 | −3.2 | 1.9 |
| Diagnosis x2 | 0.5 | 6.789 | −0.2 | 1.45 | 2.47 |
| Prescription Y | 1.2 | −0.3 | 0.4 | −0.8 | 0 |
| Procedure Z | −0.21 | −1.8 | 0.7 | −0.33333 | −4.5 |

The above representation may then be used to calculate distances between each of the clinical events (e.g., via cosine similarity, Manhattan distance, Euclidian distance, Jaccard similarity, Dice similarity, and/or the like) to generate yet another 2D data set of resulting similarities.

The resulting set of similarities may be filtered or otherwise reduced to illustrate just each clinical event's similarity to the predetermined referential clinical events as illustrated in Example Table 3 below. The similarity values previously determined may serve as a “score” for how similar each clinical event is to the referential clinical event, and a rank may be applied based upon the similarity values.

Additionally, an overall rank may be established via a suitable summary statistic, such as median rank, average score, etc. across all examples, as illustrated in Example Table 3 below.

EXAMPLE TABLE 3

| | Referential 1 | | Referential 2 | | Overall |
|---|---|---|---|---|---|
| Clinical Event | Score | Rank | Score | Rank | Rank |
| Diagnosis x1 | 0.9 | 1 | 0.7 | 1 | 1 |
| Diagnosis x2 | 0.5 | 2 | 0.1 | 3 | 2.5 |
| Diagnosis x3 | 0.4 | 3 | 0.65 | 2 | 2.5 |

In this example, the malady-treatment prediction system may determine X1 is likely to be treatable by the particular pharmaceutical, treatment and/or procedure.

### Exemplary Machine Learning Modules

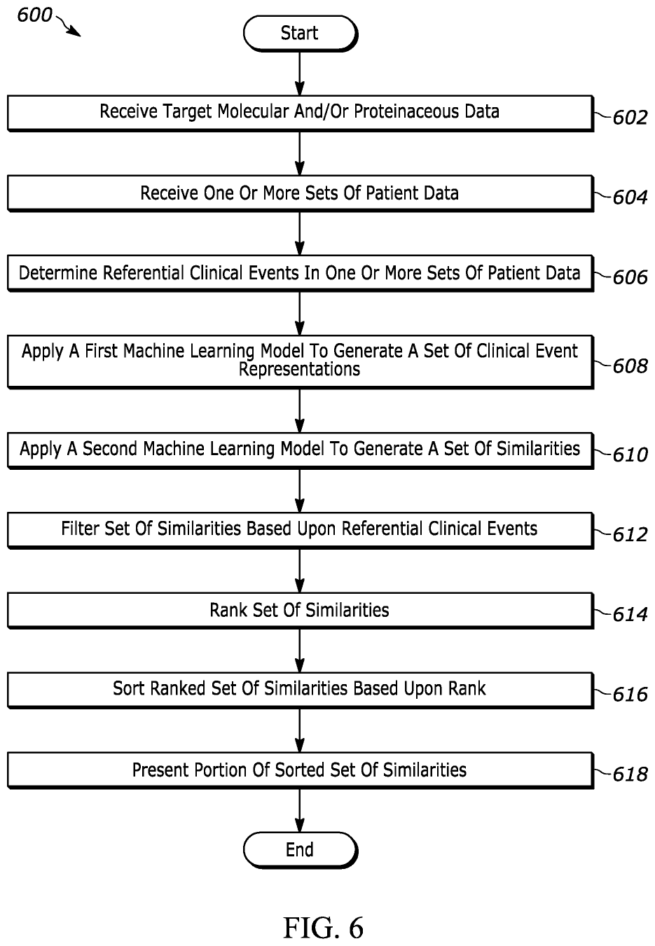

FIG. 5 depicts a diagram of exemplary machine learning modules 242. The machine learning modules 242 may include an engineering module 501, a machine learning application module 511, and/or a machine learning model 520. The engineering module 501, the machine learning application module 511, and/or the machine learning model 520 may be, or may include, a portion of a memory unit (e.g., the one or more memories 104 of FIG. 1) configured to store software and/or computer-executable instructions that, when executed by a processing unit (e.g., the one or more processors 102 of FIG. 1), may cause the one or more of the aforementioned components to generate, develop, deploy, and/or validate the machine learning model 520. There may be one or more machine learning models 520 developed by the machine learning modules 242.

The engineering module 501 may include a clinical events engineering module 502, a patient data enriching module 504, and/or a referential clinical event engineering module 506. The clinical events engineering module 502 may receive raw, real-world data from the handler module 230 and process the raw, real-world data to generate one or more sets of patient data 312 featuring patients' medical histories. The patient data enriching module 504 may further process the one or more sets of patient data 312 to determine one or more relationships and/or contexts between the various clinical events across the various patients' medical histories. The referential clinical event engineering module 506 may determine one or more referential clinical events 444 in the one or more sets of patient data from referencing a plethora of real-world data such as established drug approvals, clinical knowledge and expertise, medical literature, gene databases, and/or biomedical knowledge graphs. The determined one or more referential clinical events 444 may be a subset within the set of clinical events.

The machine learning application module 511 may include a clinical event representation engineering module 512, a similarity calculation module 514, and/or a model validation module 516. The machine learning application module 511 may receive the one or more sets of patient data 312 from the engineering module 501 and process the one or more sets of patient data 312 to generate a set of clinical event representations 322. The similarity calculation module may process the set of clinical event representations 322 to generate a set of similarities 332.

The developing machine learning model may be validated by the model validation module 516. In some embodiments, the model validation module 516 may contain a number of validation steps. For example, the model validation module 516 may use a number of positive validations derived from the result of a successful clinical trial or otherwise provided by clinical experts to validate the machine learning model 520. The model validation module 516 may determine if these positive validations rank highly when compared to the determined outputs (e.g. the positive validations rank in the top 100). If the positive validations do not rank high enough, then the machine learning model 520 may return to the engineering module 501 for further development. Conversely, if the positive validations do rank high enough, then the model validation module 516 may continue validating the machine learning model 520 until it has been sufficiently validated. As another example, the model validation module 516 may use a number of negative validations derived from the result of an unsuccessful clinical trial or otherwise provided by clinical experts to validate the machine learning model 520. The model validation module 516 may determine if these negative validations rank lowly when compared to the determined outputs (e.g. the negative validations rank below the top 200). If the negative validations do not rank low enough, then the machine learning model 520 may return to the engineering module 501 for further development. Conversely, if the negative validations do rank low enough, then the model validation module 516 may continue validating the machine learning model 520 until it has been sufficiently validated.

In some embodiments, when unsupervised machine learning is utilized, the model validation module 516 may be used to solve a related supervised machine learning problem to test the validity of the developing machine learning model. For example, the model validation module 516 may make a determination regarding the clinical outcome of a patient based upon the available patient data expressed through the learned representations (e.g., whether the patient will likely suffer a heart attack within the next six months) and this determination may be repeated for any number of patients in the patient data. In some embodiments, the resulting machine learning model 520 may be manually reviewed by a clinical expert.

Additionally or alternatively, in some embodiments, the developing machine learning model 520 may be validated by projecting representations of clinical events (e.g., diagnosed maladies) onto a 2D vector space (e.g., via principal component analysis (PCA), multidimensional scaling (MDS), uniform manifold approximation and projection (UMAP), etc.) and plot related groupings according to a hierarchical structure (e.g., the International Classification of Diseases (ICD)). Additionally or alternatively, in some embodiments, the developing machine learning model 520 may be validated by attempting to give a clinical interpretation to each categorization, classification, dimension, and/or parameter determined. For example, a determined categorization, classification, dimension, and/or parameter may be selected and the clinical events in the set of clinical event representations may be sorted according to each clinical event's individual entry under the selected categorization, classification, dimension, and/or parameter. A clinical interpretation may be assigned to a group of clinical events that have the highest values in the selected categorization, classification, dimension, and/or parameter (e.g. all clinical events related to chronic auto-immune conditions) and the lowest values in the selected categorization, classification, dimension, and/or parameter (e.g. all things related to skin diseases).

The foregoing processes may repeat until the results of the machine learning model 520 produce a satisfactory error rate. The machine learning model 520 may be updated from parallel machine learning modules 242. It should be appreciated that while specific elements, processes, devices, and/or components are described as part of example machine learning module 242, other elements, processes, devices and/or components are contemplated and/or the elements, processes, devices, and/or components may interact in different ways and/or in differing orders, etc.

### Exemplary Implementation of the Malady-Treatment Prediction System

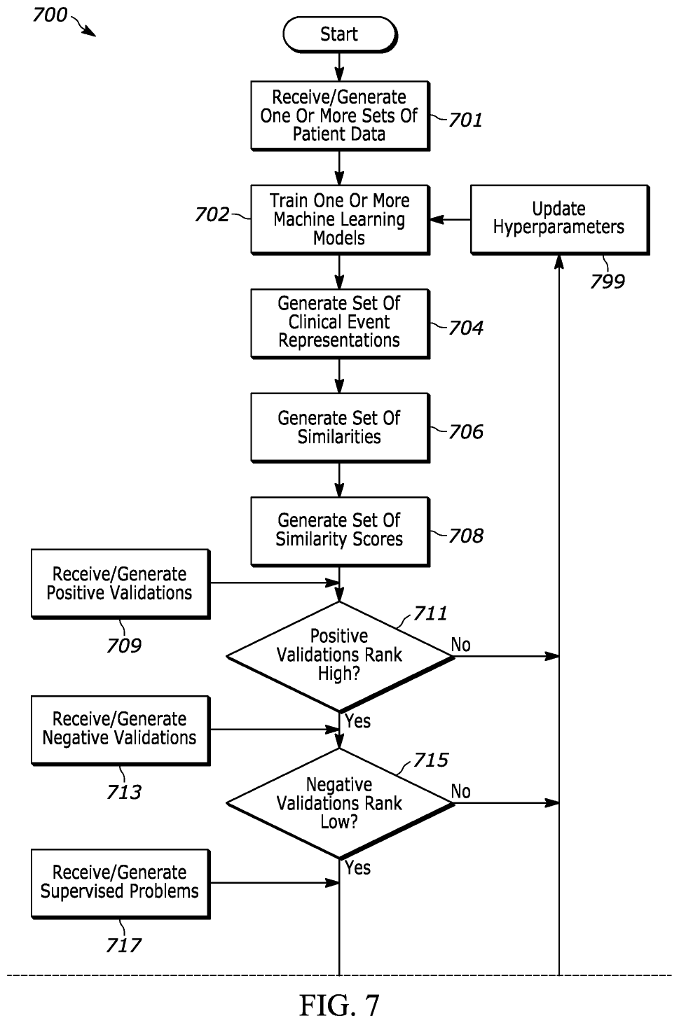

FIG. 6 depicts an exemplary computer-based method 600 for implementing a malady-treatment prediction system that may classify or predict maladies that may be treatable by pharmaceuticals, treatments, and/or procedures. In some aspects, the method 600 may correspond to, and/or be implemented by, the application server 220 of FIG. 2.

The processes, methods, software, and/or computer-executable instructions included within the method 600 may be, or may include, an executable program or portion of an executable program for execution by a processor such as the one or more processors 102 of FIG. 1. The program may be embodied in software or instructions stored on a non-transitory computer-readable storage medium or disk associated with the one or more processor 102. Further, although the example program is described with reference to the flowchart illustrated in FIG. 6, many other methods of implementing the application server 220 may alternatively be used. For example, the order of execution of the blocks may be changed, and/or some of the blocks described may be changed, eliminated, or combined.

Additionally, or alternatively, any or all of the blocks may be implemented by one or more hardware circuits (e.g., discrete and/or integrated analog and/or digital circuitry, an application specific integrated circuit (ASIC), a programmable logic device (PLD), a field programmable gate array (FPGA), a field programmable logic device (FPLD), a logic circuit, etc.) structured to perform the corresponding operation without executing software or firmware.

The method 600 of FIG. 6 may begin with a malady-treatment prediction system (e.g., application server 220) receiving target molecular and/or proteinaceous data related to a particular pharmaceutical, treatment, and/or procedure in development (block 602). Such data refers to information about molecular or protein targets or mechanisms of action, or preclinical data about its effect on different pathways. Based on how the machine learning model 520 of the malady-treatment prediction system is trained, the malady-treatment prediction system may determine, categorize, and/or predict one or more maladies that are most likely treatable by the particular pharmaceutical, treatment, and/or procedure.

The malady-treatment prediction system may receive one or more sets of patient data (block 604). The one or more sets of patient data may include (a) characteristics of the one or more patients in the set of patients, (b) one or more maladies contracted by the one or more patients, (c) one or more pharmaceuticals prescribed to the one or more patients, (d) one or more lab tests performed on the one or more patients and/or the lab tests' corresponding lab results, (e) a number of occurrences of each of the foregoing data, and/or (e) dates, timespans, and/or other chronological data of any of the foregoing data.

The malady-treatment prediction system may determine referential clinical events (e.g., specified maladies, pharmaceuticals, treatments, procedures, and/or lab tests) in the one or more sets of patient data that are related to the treatment data (block 606). The referential clinical events may be determined by referencing a plethora of real-world data such as established drug approvals, clinical knowledge and expertise, medical literature, gene databases, and/or biomedical knowledge graphs. In some embodiments, the referential clinical event may be determined based upon a threshold number of evidentiary findings that may indicate connections with a particular pharmaceutical, treatment, and/or procedure.

The malady-treatment prediction system may apply a first machine learning model (e.g., machine learning model 520a) on the one or more sets of patient data to generate a set of clinical event representations (block 608). In some embodiments, the first machine learning model is an unsupervised machine learning model. In some embodiments, the first machine learning model is a semi-supervised machine learning model (such as word2vec, BERT, other attention based-methods, and other representation learning approaches, etc.). In some embodiments, the malady-treatment prediction system may generate the set of clinical event representations by performing classical dimensionality reduction (e.g., principal component analysis (PCA), linear discriminant analysis (LDA), etc.), shifted positive pointwise mutual information (SPPMI), etc. on the one or more sets of patient data.

The malady-treatment prediction system may apply a second machine learning model (e.g., machine learning model 520b) on the set of clinical event representations to generate a set of similarities (block 610). In some embodiments, the second machine learning model is an unsupervised machine learning model. In some embodiments, the second machine learning model is a semi-supervised machine learning model. In some embodiments, the second machine learning model is the same as the first machine learning model. In some embodiments, the malady-treatment prediction system may generate the set of similarities by performing a pair-wise distance and/or similarity algorithm (e.g., cosine similarity, Euclidean distance, etc.) on each clinical event with each other clinical event.

The malady-treatment prediction system may filter the set of similarities based upon the referential clinical events (block 612). This may be done to highlight the similarities and/or differences between each clinical event and the referential clinical events.

The malady-treatment prediction system may rank the clinical events in the set of similarities based upon how similar those clinical events are to the referential clinical events (block 614).

The malady-treatment prediction system may sort the ranked set of similarities based upon each clinical event's rank to each referential clinical event (block 616).

The malady-treatment prediction system may present at least a portion of the sorted set of similarities to a client device (block 618). The method 600 may exit.

### Exemplary Implementation of the Machine Learning Training Module

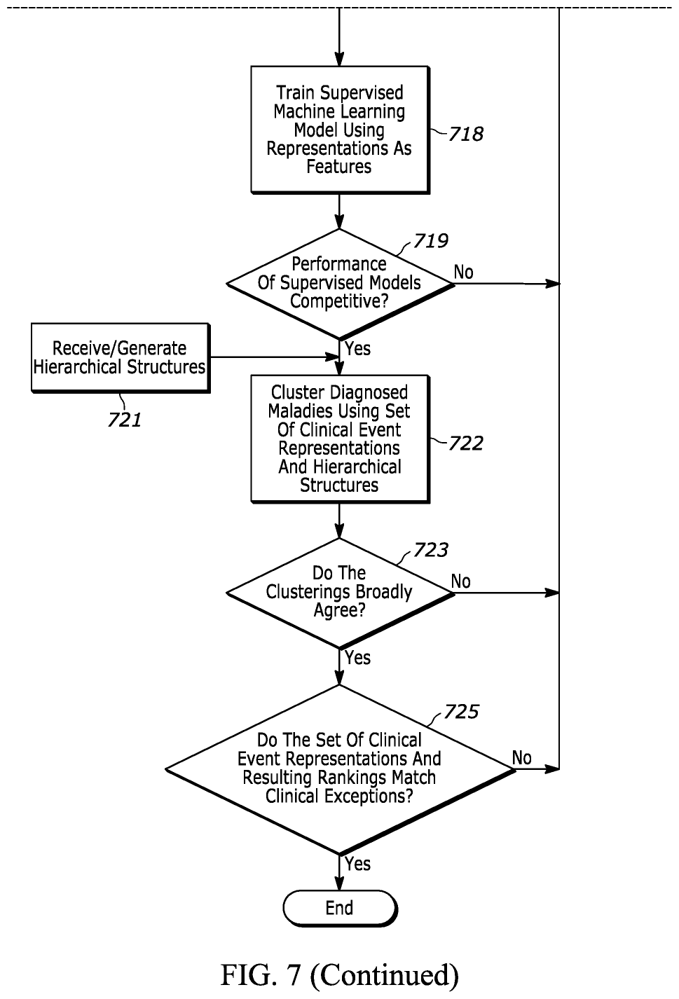

FIG. 7 depicts an exemplary computer-based method 700 for implementing the machine learning training module 700, according to some aspects. In some aspects, the method 700 may correspond to, and/or be implemented by, the engineering module 510, the machine learning model 520, and/or the machine learning application module 511 of FIG. 5.

The processes, methods, software, and/or computer-executable instructions included within the method 700 may be, or may include, an executable program or portion of an executable program for execution by a processor such as the one or more processors 102 of FIG. 1. The program may be embodied in software or instructions stored on a non-transitory computer-readable storage medium or disk associated with the one or more processor 102. Further, although the example program is described with reference to the flowchart illustrated in FIG. 7, many other methods of implementing the machine learning modules 242 may alternatively be used. For example, the order of execution of the blocks may be changed, and/or some of the blocks described may be changed, eliminated, or combined.

Additionally, or alternatively, any or all of the blocks may be implemented by one or more hardware circuits (e.g., discrete and/or integrated analog and/or digital circuitry, an application specific integrated circuit (ASIC), a programmable logic device (PLD), a field programmable gate array (FPGA), a field programmable logic device (FPLD), a logic circuit, etc.) structured to perform the corresponding operation without executing software or firmware.

The method 700 of FIG. 7 may begin upon receiving and/or generating one or more sets of patient data (block 701). The method 700 may use the one or more sets of patient data to train one or more machine learning models (block 702). Once trained, the machine learning models may generate a set of clinical event representations using the one or more sets of patient data (block 704) and generate a set of similarities using the set of clinical event representations (block 706). The method 700 may filter the set of similarities using one or more predetermined referential clinical events to generate a set of similarity scores (block 708).

The method 700 may receive, determine, and/or generate one or more positive validations (e.g., one or more maladies validated as treatable by one or more clinical trials) (block 709). Upon receiving the one or more positive validations, the method 700 may determine whether the positive validations rank above a predetermined threshold (block 711). If the positive validations do not include values that exceed the predetermined threshold (block 711), the method 700 may update hyperparameters of the one or more machine learning models (block 750) and return back to block 702. In some embodiments, hyperparameters may include elements relating to (i) the run time the one or more machine learning models, (ii) the degree of variance between elements in the architecture of the one or more machine learning models, and/or (iii) any other parameters required by the model.

If a sufficient proportion of the positive validations rank above the predetermined threshold (block 711), the method 700 may proceed by receiving, determining, and/or generating one or more negative validations (e.g., one or more maladies validated as not treatable by one or more clinical trials) (block 713). Upon receiving the one or more negative validations, the method 700 may determine whether the negative validations rank below a predetermined threshold (block 715). If the negative validations do not include values that do not meet or do not exceed the predetermined threshold (block 715), the method 700 may update hyperparameters of the one or more machine learning models (block 750) and return back to block 702.

If the negative validations do not meet or do not exceed a predetermined the threshold (block 715), the method 700 may proceed by receiving, determining, and/or generating one or more supervised machine learning problems (such as a prediction as to whether a patient will suffer from heat stroke in the next 6 months) (block 717). Upon receiving the one or more supervised machine learning problems, the method 700 may train one or more supervised machine learning models using the set of clinical event representations as features (block 718). The method may determine whether the one or more supervised machine learning models are competitive (block 719). If the one or more supervised machine learning models are not competitive (block 719), the method 700 may update hyperparameters of the one or more machine learning models (block 750) and return back to block 702.

If the one or more supervised machine learning models are competitive (719), the method 700 may proceed by receiving, determining, and/or generating one or more hierarchical structures (e.g., the International Classification of Diseases (ICD)) (block 721). The method 700 may cluster the diagnosed malady clinical events using the set of clinical event representations and the one or more hierarchical structures (block 722). The method 700 may determine whether the resulting clusterings broadly agree (block 723). If the resulting clusterings do not broadly agree (block 723), the method 700 may update hyperparameters of the one or more machine learning models (block 750) and return back to block 702.

If the resulting clusterings do broadly agree (block 723), the method 700 may proceed by determining whether the set of clinical event representations, in addition to the resulting rankings, match clinical expectations (block 725). If the set of clinical event representations, in addition to the resulting rankings, do not match clinical expectations (block 725), the method 700 may update hyperparameters of the one or more machine learning models (block 750) and return back to block 702. If the set of clinical event representations, in addition to the resulting rankings, do match clinical expectations (block 725), the method 700 may exit. It should be noted that method 700 is illustrative in nature and that development and validation of the one or more machine learning models include additional, less, or alternate actions, and/or actions presented in an alternate order, including those discussed elsewhere herein.

### Exemplary Method

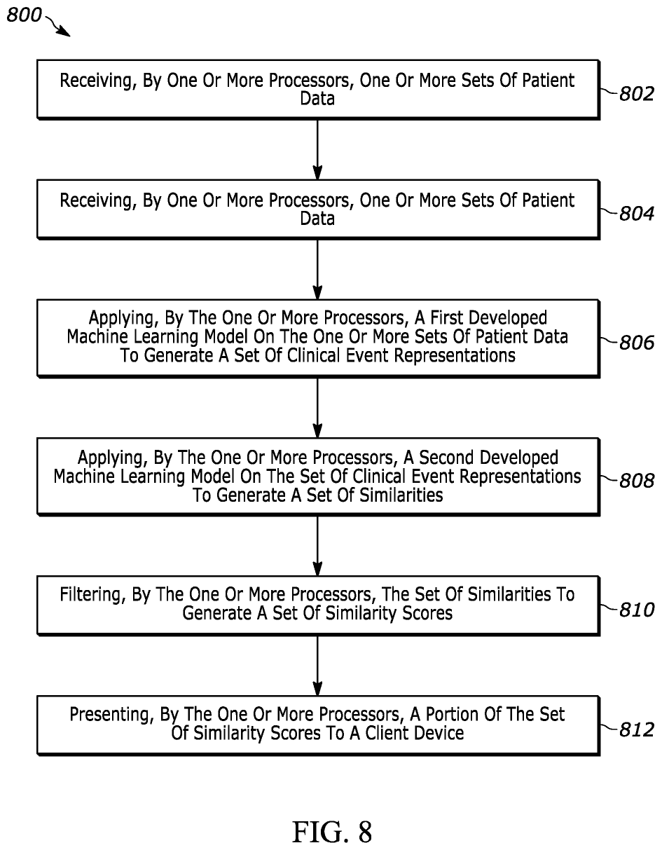

FIG. 8 depicts an exemplary artificial intelligence based method 800 for classifying or predicting maladies that may be treatable by pharmaceuticals, treatments, and/or procedures. The method 800 depicted in FIG. 7 may employ any of the techniques, methods, and systems described herein with respect to FIGS. 1-6.

The method 800 may begin at block 802 by receiving, by one or more processors, one or more sets of patient data. In some embodiments, the one or more sets of patient data may be segmented into one or more subsets by each patient and each segmented subset may include a timeline of one or more clinical events. The clinical events may include one or more maladies diagnosed on each patient, one or more pharmaceuticals prescribed to each patient, one or more procedures performed on each patient, and/or one or more tests performed on each patient.

The method 800 may proceed to block 804 by determining, by the one or more processors, one or more referential clinical events in the one or more sets of patient data. In some embodiments, the referential clinical events may be one or more data points derived from the patient data based upon one or more sources of real-world data (e.g., established drug approvals, clinical knowledge and expertise, medical literature, gene databases, and/or biomedical knowledge graphs). The referential clinical events may be determined based upon a threshold number of evidentiary findings that may indicate connections with a particular pharmaceutical, treatment, and/or procedure.

The method 800 may proceed to block 806 by applying, by the one or more processors, a first developed machine learning model on the one or more referential clinical events to generate a set of clinical event representations. In some embodiments, the set of clinical event representations may be determined by semi-supervised learning methods and attention methods, dimensionality reduction, anomaly detection, low-density separation, Laplacian regularization, neural networks, deep learning, and/or any other suitable machine learning technique. In some embodiments, the machine learning model may be trained via unsupervised learning and/or semi-supervised learning. In these embodiments, the machine learning model may use the input data to determine parameters and/or categories of similarity. The determined parameters and/or categories of similarity may then be used to compare the individual clinical events of the input data in the set of clinical event representations against each other clinical event in the set of clinical event representations. The one or more sets of patient data and/or the set of clinical event representations may also include one or more referential clinical events.

The method 800 may proceed to block 808 by applying, by the one or more processors, a second developed machine learning model on the set of clinical event representations to generate a set of similarities, wherein the one or more sets of similarities include a determined similarity value of each clinical event in the set of clinical event representations across each other clinical event in the set of clinical event representations. A machine learning module (e.g., machine learning module 242) may generate a machine learning model (e.g., machine learning model 520) based upon training data. The machine learning module may then validate the resulting machine learning model generated. In some embodiments, the first developed machine learning model is the same as the second developed machine learning model.

In some embodiments, the second developed machine learning model may perform a distance and/or similarity algorithm on the one or more clinical events based upon the determined categorizations, classifications, dimensions, and/or parameters. For example, the second developed machine learning model may perform pair-wise similarity between each of the clinical event representations of each patient in input data to determine the set of similarities between each of the clinical events. Any appropriate distance and/or similarity algorithm may be utilized to generate the set of similarities, such as cosine similarity, Manhattan distance, Euclidian distance, Jaccard similarity, Dice similarity, and/or the like.

The method 800 may proceed to block 810 by filtering, by the one or more processors, the set of similarities to generate a set of similarity scores. Upon generating the set of similarities, the malady-treatment prediction system may filter out the set of similarities set based upon one or more predetermined referential clinical events. To put another way, the malady-treatment prediction system may focus on the portion of the resulting set of similarities to illustrate how similar each clinical event is to the one or more referential clinical events. The referential clinical events may be predetermined by established drug approvals, clinical knowledge and expertise, medical literature, gene databases, and/or biomedical knowledge graphs.

The method 800 may proceed to block 812 by presenting, by the one or more processors, at least a portion of the set of similarities to a client device.

The method 800 may include additional, less, or alternate actions, and/or actions presented in an alternate order, including those discussed elsewhere herein.

### Additional Exemplary Embodiments Malady-Treatment Prediction System

In one aspect, an artificial intelligence based method for classifying or predicting maladies that may be treatable by pharmaceuticals, treatments, and/or procedures may be provided. The method may be implemented via one or more local and/or remote processors, transceivers, sensors, servers, memory units, mobile devices, wearables, smart glasses, augmented reality glasses, virtual reality headsets, and/or other electronic and/or electrical components. In one instance, the method may include: (1) receiving, by one or more processors, one or more sets of patient data; (2) determining, by the one or more processors, one or more referential clinical events in the one or more sets of patient data; (3) applying, by the one or more processors, a first developed machine learning model on the one or more referential clinical events to generate a set of clinical event representations; (4) applying, by the one or more processors, a second developed machine learning model on the set of clinical event representations to generate a set of similarities; (5) filtering, by the one or more processors based upon one or more predetermined referential clinical events, the set of similarities to generate a set of similarity scores; and/or (6) presenting, by the one or more processors, at least a portion of the set of similarities to a client device. The method may include additional, less, or alternate actions, including those discussed elsewhere herein.

Additionally or alternatively to the foregoing method, the method may further include ranking, by the one or more processors, the set of similarities based upon the generated set of similarity scores; sorting, by the one or more processors, the set of similarities based upon the ranking of the set of similarities; and/or presenting, by the one or more processors, a portion of the sorted set of similarities to the client device. In some embodiments, the method may further include estimating, by the one or more processors, a degree of uncertainty of the set of similarity scores and/or adjusting, by the one or more processors, one or more of (i) the first developed machine learning model or (ii) the second developed machine learning model based on (a) the set of similarity scores and (b) the estimated degree of uncertainty to minimize the degree of uncertainty of the set of similarity scores. Additionally or alternatively to the foregoing method, (i) the one or more sets of patient data includes one or more subsets segmented by patient and each segmented subset may include a timeline of one or more clinical events, the one or more clinical events may include: one or more maladies diagnosed on each patient, one or more pharmaceuticals prescribed to each patient, one or more procedures performed on each patient, or one or more tests performed on each patient, (ii) the set of clinical event representations may include one or more determined dimensions of the one or more clinical events, and/or (iii) wherein the one or more sets of similarities may include a determined similarity value of each clinical event in the set of clinical event representations across each other clinical event in the set of clinical event representations.

Additionally or alternatively to the foregoing method, the one or more determined dimensions of the set of clinical event representations may be generated via dimensionality reduction. Additionally or alternatively to the foregoing method, the method may further include: receiving, by the one or more processors, a set of target molecular/proteinaceous data (see FIG. 6), wherein at least one of (i) the first developed machine learning model or (ii) the second developed machine learning model is applied to the set of target molecular/proteinaceous data.

Additionally or alternatively to the foregoing method, the method may further include: receiving, by the one or more processors, a set of real world data; training, by the one or more processors, a third developed machine learning model to generate an accuracy determination of one or more unsupervised machine learning models using a training data set of previously generated sets of similarities as training data; validating, by the one or more processors, the third developed machine learning model using the set of real world data as validation data; inputting, by the one or more processors, the set of similarities into the third developed machine learning model to generate an accuracy determination of at least one of (i) the first developed machine learning model or (ii) the second developed machine learning model; and/or adjusting, by the one or more processors, at least one of (i) the first developed machine learning model or (ii) the second developed machine learning model based on the accuracy determination of the third developed machine learning model.

In another aspect, a computer system for classifying or predicting maladies that may be treatable by pharmaceuticals, treatments, and/or procedures may be provided. The computer system may be configured to include one or more local and/or remote processors, transceivers, sensors, servers, memory units, mobile devices, wearables, smart glasses, augmented reality glasses, virtual reality headsets, and/or other electronic and/or electrical components. In one instance, the computer system may include one or more processors (e.g., the one or more processors 102); and/or a non-transitory program memory (e.g., the one or more memories 104) coupled to the one or more processors and/or storing executable instructions that, when executed by the one or more processors, cause the computer system to: (1) receive one or more sets of patient data; (2) determine one or more referential clinical events in the one or more sets of patient data; (3) apply a first developed machine learning model on the one or more referential clinical events to generate a set of clinical event representations; (4) apply a second developed machine learning model on the set of clinical event representations to generate a set of similarities; (5) filter, based upon one or more predetermined referential clinical events, the set of similarities to generate a set of similarity scores; and/or (6) present at least a portion of the set of similarities to a client device. The computer system may be configured to include additional, less, or alternate functionality, including that discussed elsewhere herein.

Additionally or alternatively to the foregoing system, the instructions may further cause the system to: rank the set of similarities based upon the generated set of similarity scores; sort the set of similarities based upon the ranking of the set of similarities; and/or present a portion of the sorted set of similarities to the client device. In some embodiments, the instructions may further cause the system to estimate a degree of uncertainty of the set of similarity scores and/or adjust one or more of (i) the first developed machine learning model or (ii) the second developed machine learning model based on (a) the set of similarity scores and (b) the estimated degree of uncertainty to minimize the degree of uncertainty of the set of similarity scores. Additionally or alternatively to the foregoing system, (i) the one or more sets of patient data includes one or more subsets segmented by patient and each segmented subset may include a timeline of one or more clinical events, the one or more clinical events may include: one or more maladies diagnosed on each patient, one or more pharmaceuticals prescribed to each patient, one or more procedures performed on each patient, or one or more tests performed on each patient, (ii) the set of clinical event representations may include one or more determined dimensions of the one or more clinical events, and/or (iii) wherein the one or more sets of similarities may include a determined similarity value of each clinical event in the set of clinical event representations across each other clinical event in the set of clinical event representations.

Additionally or alternatively to the foregoing system, the one or more determined dimensions of the set of clinical event representations may be generated via dimensionality reduction. Additionally or alternatively to the foregoing system, the instructions may further cause the system to: receive a set of target molecular/proteinaceous data, wherein at least one of (i) the first developed machine learning model or (ii) the second developed machine learning model is applied to the set of target molecular/proteinaceous data.

Additionally or alternatively to the foregoing system, the instructions may further cause the system to: receive a set of real world data; train a third developed machine learning model to generate an accuracy determination of one or more unsupervised machine learning models using a training data set of previously generated sets of similarities as training data; validate the third developed machine learning model using the set of real world data as validation data; input the set of similarities into the third developed machine learning model to generate an accuracy determination of at least one of (i) the first developed machine learning model or (ii) the second developed machine learning model; and/or adjust at least one of (i) the first developed machine learning model or (ii) the second developed machine learning model based on the accuracy determination of the third developed machine learning model.

In another aspect, a tangible, a non-transitory computer-readable medium may store executable instructions for classifying or predicting maladies that may be treatable by pharmaceuticals, treatments, and/or procedures may be provided. The executable instructions, when executed, may cause one or more processors (e.g., the one or more processors 102) to: (1) receive one or more sets of patient data; (2) determine one or more referential clinical events in the one or more sets of patient data; (3) apply a first developed machine learning model on the one or more referential clinical events to generate a set of clinical event representations; (4) apply a second developed machine learning model on the set of clinical event representations to generate a set of similarities; (5) filter, based upon one or more predetermined referential clinical events, the set of similarities to generate a set of similarity scores; and/or (6) present at least a portion of the set of similarities to a client device. The instructions may direct additional, less, or alternate functionality, including that discussed elsewhere herein.

Additionally or alternatively to the foregoing executable instructions, the executable instructions may further cause the one or more processors to: rank the set of similarities based upon the generated set of similarity scores; sort the set of similarities based upon the ranking of the set of similarities; and/or present a portion of the sorted set of similarities to the client device. In some embodiments, the instructions may further cause the one or more processors to estimate a degree of uncertainty of the set of similarity scores and/or adjust one or more of (i) the first developed machine learning model or (ii) the second developed machine learning model based on (a) the set of similarity scores and (b) the estimated degree of uncertainty to minimize the degree of uncertainty of the set of similarity scores. Additionally or alternatively to the foregoing executable instructions, (i) the one or more sets of patient data includes one or more subsets segmented by patient and each segmented subset may include a timeline of one or more clinical events, the one or more clinical events may include: one or more maladies diagnosed on each patient, one or more pharmaceuticals prescribed to each patient, one or more procedures performed on each patient, or one or more tests performed on each patient, (ii) the set of clinical event representations may include one or more determined dimensions of the one or more clinical events, and/or (iii) wherein the one or more sets of similarities may include a determined similarity value of each clinical event in the set of clinical event representations across each other clinical event in the set of clinical event representations.

Additionally or alternatively to the foregoing executable instructions, the one or more determined dimensions of the set of clinical event representations may be generated via dimensionality reduction. Additionally or alternatively to the foregoing executable instructions, the executable instructions may further cause the one or more processors to: receive a set of target molecular/proteinaceous data, wherein at least one of (i) the first developed machine learning model or (ii) the second developed machine learning model is applied to the set of target molecular/proteinaceous data.

Additionally or alternatively to the foregoing executable instructions, the executable instructions may further cause the one or more processors to: receive a set of real world data; train a third developed machine learning model to generate an accuracy determination of one or more unsupervised machine learning models using a training data set of previously generated sets of similarities as training data; validate the third developed machine learning model using the set of real world data as validation data; input the set of similarities into the third developed machine learning model to generate an accuracy determination of at least one of (i) the first developed machine learning model or (ii) the second developed machine learning model; and/or adjust at least one of (i) the first developed machine learning model or (ii) the second developed machine learning model based on the accuracy determination of the third developed machine learning model.

### ADDITIONAL CONSIDERATIONS

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, some embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more modules of a computer system (e.g., a processor or a group of processors) may be configured by software (e.g., an application or application portion) as a module that operates to perform certain operations as described herein.

In various embodiments, a module may be implemented mechanically or electronically. Accordingly, the term “module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which modules are temporarily configured (e.g., programmed), each of the modules need not be configured or instantiated at any one instance in time. For example, where the modules comprise a general-purpose processor configured using software, the general-purpose processor may be configured as respective different modules at different times. Software may accordingly configure a processor, for example, to constitute a particular module at one instance of time and to constitute a different module at a different instance of time.

Modules may provide information to, and receive information from, other modules. Accordingly, the described modules may be regarded as being communicatively coupled. Where multiple of such modules exist contemporaneously, communications may be achieved through signal transmission (e.g., over appropriate circuits and buses) that connect the modules. In embodiments in which multiple modules are configured or instantiated at different times, communications between such modules may be achieved, for example, through the storage and retrieval of information in memory structures to which the multiple modules have access. For example, one module may perform an operation and store the output of that operation in a memory device to which it is communicatively coupled. A further module may, at a later time, access the memory device to retrieve and process the stored output. Modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

Unless specifically stated otherwise, discussions herein using words such as “receiving,” “analyzing,” “generating,” “creating,” “storing,” “deploying,” “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information. Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other.

As used herein any reference to “some embodiments” means that a particular element, feature, structure, or characteristic described in connection with the embodiment may be included in at least one embodiment. The appearances of the phrase “some embodiments” in various places in the specification are not necessarily all referring to the same embodiment. In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

This detailed description is to be construed as exemplary only and does not describe every possible embodiment, as describing every possible embodiment would be impractical, if not impossible. One could implement numerous alternate embodiments, using either current technology or technology developed after the filing date of this application. Upon reading this disclosure, those of skill in the art will appreciate still additional alternative structural and functional designs for the systems and methods disclosed herein.

Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein. It is therefore intended that the foregoing detailed description be regarded as illustrative rather than limiting, and that it be understood that it is the following claims, including all equivalents, that are intended to define the spirit and scope of this invention.

What is claimed is:

1. An artificial intelligence based method for classifying or predicting maladies that may be treatable by pharmaceuticals, treatments, and/or procedures, the artificial intelligence based method comprising:
receiving, by one or more processors, one or more sets of patient data;
determining, by the one or more processors, one or more clinical events in the one or more sets of patient data;
applying, by the one or more processors, a first developed machine learning model on the one or more clinical events to generate a set of clinical event representations;
applying, by the one or more processors, a second developed machine learning model on the set of clinical event representations to generate a set of similarities;
filtering, by the one or more processors based upon one or more predetermined clinical events, the set of similarities to generate a set of similarity scores; and
presenting, by the one or more processors, at least a portion of the set of similarities to a client device.

2. The artificial intelligence based method of claim 1, further comprising:
ranking, by the one or more processors, the set of similarities based upon the generated set of similarity scores;
sorting, by the one or more processors, the set of similarities based upon the ranking of the set of similarities; and
presenting, by the one or more processors, a portion of the sorted set of similarities to the client device.

3. The artificial intelligence based method of claim 2, further comprising:
estimating, by the one or more processors, a degree of uncertainty of the set of similarity scores; and
adjusting, by the one or more processors, one or more of (i) the first developed machine learning model or (ii) the second developed machine learning model based on (a) the set of similarity scores and (b) the estimated degree of uncertainty to minimize the degree of uncertainty of the set of similarity scores.

4. The artificial intelligence based method of claim 1, wherein:
the one or more sets of patient data includes one or more subsets segmented by patient and each segmented subset includes a timeline of one or more clinical events.

5. The artificial intelligence based method of claim 1, wherein:
the one or more clinical events include one or more maladies diagnosed on each patient, one or more pharmaceuticals prescribed to each patient, one or more procedures performed on each patient, or one or more tests performed on each patient.

6. The artificial intelligence based method of claim 1, wherein:
the set of clinical event representations includes one or more determined dimensions for the one or more clinical events.

7. The artificial intelligence based method of claim 1, wherein:
wherein the one or more sets of similarities include a determined similarity value of each clinical event in the set of clinical event representations across each other clinical event in the set of clinical event representations.

8. The artificial intelligence based method of claim 6, wherein:
the one or more determined dimensions of the set of clinical event representations are generated via dimensionality reduction or representation learning, and
the method further comprising:
receiving, by the one or more processors, data related to a particular pharmaceutical, treatment, and/or procedure in development, wherein at least one of (i) the first developed machine learning model or (ii) the second developed machine learning model is applied to the data.

9. A computer system for classifying or predicting maladies that may be treatable by pharmaceuticals, treatments, and/or procedures, the computer system comprising:
one or more processors; and
one or more non-transitory program memories coupled to the one or more processors, the one or more memories storing executable instructions that, when executed by the one or more processors, cause the one or more processors to:
receive one or more sets of patient data;
determine one or more clinical events in the one or more sets of patient data;
apply a first developed machine learning model on the one or more clinical events to generate a set of clinical event representations;
apply a second developed machine learning model on the set of clinical event representations to generate a set of similarities;
filter, based upon one or more predetermined clinical events, the set of similarities to generate a set of similarity scores; and
present at least a portion of the set of similarities to a client device.

10. The computer system of claim 9, wherein the executable instructions, when executed by the one or more processors, further cause the one or more processors to:
rank the set of similarities based upon the generated set of similarity scores;
sort the set of similarities based upon the ranking of the set of similarities; and
present a portion of the sorted set of similarities to the client device.

11. The computer system of claim 10, wherein the executable instructions, when executed by the one or more processors, further cause the one or more processors to:
estimate a degree of uncertainty of the set of similarity scores; and
adjust one or more of (i) the first developed machine learning model or (ii) the second developed machine learning model based on (a) the set of similarity scores and (b) the estimated degree of uncertainty to minimize the degree of uncertainty of the set of similarity scores.

12. The computer system of claim 9, wherein:
the one or more sets of patient data includes one or more subsets segmented by patient and each segmented subset includes a timeline of one or more clinical events.

13. The computer system of claim 9, wherein:
the one or more clinical events include one or more maladies diagnosed on each patient, one or more pharmaceuticals prescribed to each patient, one or more procedures performed on each patient, or one or more tests performed on each patient.

14. The computer system of claim 9, wherein:
the set of clinical event representations includes one or more determined dimensions for the one or more clinical events.

15. The computer system of claim 9, wherein:
wherein the one or more sets of similarities include a determined similarity value of each clinical event in the set of clinical event representations across each other clinical event in the set of clinical event representations.

16. The computer system of claim 14, wherein:
the one or more determined dimensions of the set of clinical event representations are generated via dimensionality reduction or representation learning, and
wherein executable instructions that, when executed by the one or more processors, further cause the one or more processors to:
receive, by the one or more processors, data related to a particular pharmaceutical, treatment, and/or procedure in development, wherein at least one of (i) the first developed machine learning model or (ii) the second developed machine learning model is applied to the data.

17. A tangible, non-transitory computer-readable medium storing executable instructions for classifying or predicting maladies that may be treatable by pharmaceuticals, treatments, and/or procedures, the instructions, when executed by one or more processors, cause the one or more processors to:
receive one or more sets of patient data;
determine one or more clinical events in the one or more sets of patient data;
apply a first developed machine learning model on the one or more clinical events to generate a set of clinical event representations;
apply a second developed machine learning model on the set of clinical event representations to generate a set of similarities;
filter, based upon one or more predetermined clinical events, the set of similarities to generate a set of similarity scores; and
present at least a portion of the set of similarities to a client device.

18. The tangible, non-transitory computer-readable medium of claim 17, wherein the instructions, when executed by the one or more processors, further cause the one or more processors to:
rank the set of similarities based upon the generated set of similarity scores;
sort the set of similarities based upon the ranking of the set of similarities; and
present a portion of the sorted set of similarities to the client device.

19. The tangible, non-transitory computer-readable medium of claim 18, wherein the executable instructions, when executed by the one or more processors, further cause the one or more processors to:
estimate a degree of uncertainty of the set of similarity scores; and
adjust one or more of (i) the first developed machine learning model or (ii) the second developed machine learning model based on (a) the set of similarity scores and (b) the estimated degree of uncertainty to minimize the degree of uncertainty of the set of similarity scores.

20. The tangible, non-transitory computer-readable medium of claim 17, wherein:
the one or more sets of patient data includes one or more subsets segmented by patient and each segmented subset includes a timeline of one or more clinical events.

21. The tangible, non-transitory computer-readable medium of claim 17, wherein:
the one or more clinical events include one or more maladies diagnosed on each patient, one or more pharmaceuticals prescribed to each patient, one or more procedures performed on each patient, or one or more tests performed on each patient.

22. The tangible, non-transitory computer-readable medium of claim 17, wherein:
the set of clinical event representations includes one or more determined dimensions for the one or more clinical events.

23. The tangible, non-transitory computer-readable medium of claim 17, wherein:
wherein the one or more sets of similarities include a determined similarity value of each clinical event in the set of clinical event representations across each other clinical event in the set of clinical event representations.

24. The tangible, non-transitory computer-readable medium of claim 22, wherein:
the one or more determined dimensions of the set of clinical event representations are generated via dimensionality reduction or representation learning, and
wherein executable instructions that, when executed by the one or more processors, further cause the one or more processors to:
receive, by the one or more processors, data related to a particular pharmaceutical, treatment, and/or procedure in development, wherein at least one of (i) the first developed machine learning model or (ii) the second developed machine learning model is applied to the data.
